(19) United States
(12) Patent Application Publication
(10) Pub. No.: US 2024/0115406 A1
(43) Pub. Date: Apr. 11, 2024

(54) CRIMPING OF FIBROUS IMPLANTS BY HEAT TREATMENT

(71) Applicant: Stentit BV, Eindhoven (NL)

(72) Inventors: Bart SANDERS, Eindhoven (NL); Maria, Alida, Antonia TROMMELEN, Eindhoven (NL)

(21) Appl. No.: 18/263,680

(22) PCT Filed: Feb. 7, 2022
(86) PCT No.: PCT/EP2022/052872

§ 371 Date: Jul. 31, 2023

(30) Foreign Application Priority Data
Feb. 8, 2021 (EP) 21155888.7

(51) Int. Cl. A61F 2/95; A61F 2/958; B29C 71/02

(52) U.S. Cl. CPC A61F 2/9522; A61F 2/958; B29C 71/02; A61F 2240/001; B29C 2071/022; B29L 2031/7543

(57) ABSTRACT

The present invention relates to fibrous medical devices; more particularly, this invention relates to processes for crimping a fibrous tubular implant onto a support or delivery device using heat treatment.

The present invention relates to a method of making a medical device and further to a medical device comprising a fibrous structure.

In particular, the present invention relates to fibrous medical devices; more particularly, this invention relates to processes for crimping a fibrous tubular implant onto a support or delivery device using heat treatment.

Stents are being used to treat patients suffering from cardiovascular diseases, where such implants are capable of for instance; opening vascular occlusions, seal lesions, deliver drugs to specific target sites, enable endovascular delivery of valves or membranes and can even be used to shield aneurysms to prevent rupture.

Stents are mainly delivered minimally invasively using an intraluminal intervention. Therefore, those implants can undergo a geometrical transition inside the host, going from a smaller to a larger diameter. In this way, the implant can be delivered through a small incision, and be deployed inside the vasculature to adhere and/or overstretch the native artery to stay in place. A certain subclass of stents, the so called “balloon expandable stents” can be positioned in the body by making use of balloon catheters. Therefore, the stent needs to be firmly mounted onto the delivery balloon during the implantation procedure. Once mounted, the balloon catheter onto which the implant in mounted, is being inserted into the body, and guided through the vasculature to the target side. At the right location, the balloon can be pressurized by which the balloon will inflate. In this way, the pressurized balloon can push the implant open to enforce diameter enlargement, securing the positioning of the implant into the vasculature.

Proper mounting of the stent on the balloon is needed to prevent dislodgement of the stent during the implantation procedure, which is normally being done using a method called crimping. Here, balloon expandable stents are normally made from solid wall tubes, by which distinct stent patterns can be created by using for instance laser cutting. Normally, the diameter of this solid wall tube defines the nominal diameter of the stent. The nominal diameter of the stent is always larger than the deflated, and folded crossing profile of the balloon. To mount the stent onto the balloon, the stent therefore needs to undergo a crimping treatment by which the nominal stent diameter is being reduced to meet the diameter of the deflated balloon on the catheter. First, the stent will be precisely positioned over the balloon with respect to the markers on the catheter. Once in place, the stent is being mechanically crimped by which external compression firmly mounts the stent onto the pre-folded balloon. This step may involve a heat treatment under compression to relieve internal strains and stresses in the stent material and prevent recoil.

Within the field of regenerative medicine, fibrous implants are increasingly being used in for instance cardiovascular applications. They provide the capacity for host cells to interact with a network composed of fibers, to trigger a regenerative response aiming to restore tissue components. Recent inventions are exploring the possibility to use fibrous tubular implants for stenting applications (WO2017118755A1). These fibrous conduits are made out of a network composed of micro and/or nanofibers, in which balloon inflation induces rearrangement of this fibrous network to accommodate for diameter enlargement. Downside of this approach is that the lack of a distinct stent pattern in such constructs, limits the ability to mechanically crimp the stent on a balloon. Here, in those fibrous conduits, there are no macroscopic voids present that would allow the stent struts to move toward each other and reduce the diameter of the conduit as in any other conventional stent crimping approach. Instead, applying elevated external radial force required for crimping, would lead to a buildup of stress causing a limited diameter decrease, and eventually buckling of such fibrous stents.

U.S. Pat. No. 8,052,912 B2 describes a temperature controlled crimping method for a polymeric stent.

Abhari et al. describe the effect of annealing on the mechanical properties and the degradation of electrospun polydioxanone filaments (Abhari et al., Journal of the mechanical behavior of biomedical materials 67 (2017), p. 127-134).

Li et al., describe the development of eco-efficient micro-porous membranes via electrospinning and annealing of poly lactic acid (PLA) (Li et al, Journal of Membrane Science 436 (2013), p. 57-67).

It is an object of the present invention to describe and provide new and alternative ways for heat treatments (so-called annealing) for medical devices with fibrous structure, especially in that the mechanical properties of such structures can be enhanced and that an improved solution for fixation of e.g. a stent with such a fibrous structure on a balloon catheter can be provided.

The invention is defined by the independent claims. Further embodiments are subject-matter of the dependent claims.

In particular, according to the present invention a method of making a medical device is provided, comprising at least the following steps:
Providing a fibrous structure; and
annealing said fibrous structure.

The invention is based on the general idea and concept that by making use of the intrinsic crimping behavior of fibrous conduits upon annealing, such conduits could be crimped over a balloon catheter system to firmly mount the stent to balloon and prevent stent dislodgement while performing the implantation procedure.

In this way, the present invention advances the art by crimping fibrous tubular conduits onto balloon catheter systems, by exposing the construct to a heat treatment such as an annealing procedure, to induce crimp of the fibrous implant and thereby fixating the conduit over a delivery balloon, without needing to use conventional stent crimping devices.

The fibrous structure is preferably made of a network of polymeric fibers in the micrometric or nanometric range. Reference is made especially to U.S. Pat. No. 10,813,777 B2.

Annealing in the context of this disclosure is especially to be understood as a process where objects are exposed to elevated temperatures. This elevated temperature might also have an additional effect when applying it to electrospun constructs. Electrospinning in the context of this disclosure is especially to be understood as a process where polymer material is dissolved in a solvent. While applying a voltage difference over a collector and a polymer solution, polymeric jets can be drawn from such solutions. Over the distance of these jets when being drawn from the solution towards the collector or rotating mandrel, the solvent will evaporate. This will result in solidification of the polymeric material. As the polymer chains will be locked in a short period of time, stress and strain can be build up in the polymeric molecular chains as well as inside the polymer fibers and even the entire fibrillated construct or conduit. An annealing step will aid to enable the relaxation of these polymeric molecular chains, fibers and network to release the build up of stress and strain.

Another feature of annealing is that it may induce shape-memory. In case of electrospinning, annealing would release the stress and strains that was build up in the construct during processing. If than a load it applied to induce stretch on the object, new stress and strain will build up in the construct. When releasing the load causing the stretch, the structure wants to go back to its most energetic stable configuration, being it the initial state right after annealing and before stretching. If however, the electrospun construct would have been annealed while being under stretch, the stresses and strains that the stretch caused to the object would dissipate. If than after the annealing step one would remove the load that caused the stretch, the construct would remain in its stretched configuration. Hereby, the object gained a new “shape-memory”.

When electro spun conduits are being used for stent applications (WO2017118755A1), it may improve the performance of the implant to anneal the conduit at the desired diameter for application. This would mean, if the stent is indented to be placed inside a 3 mm artery, to anneal the conduit at 3 mm or a bit oversized, to induce the shape memory. If than later the conduit is being crimped or compressed for implantation, the implant will go back to its shape memory to further expand inside the artery. This will limit potential recoil of the stent.

Another additional effect of applying an annealing step to electrospun conduits, is that the elevated temperature might support the evaporation of potential solvents still residing within the conduit after electrospinning. It may be that during the electrospinning process, not all the solvent has fully evaporated while forming the polymer fiber when being drawn from the solution. Even small particles might still be trapped within the polymer fibers. Here, the elevated temperature will heat the construct to ease the evaporation of the solvent. Hereby, the crystallinity of the polymer fibers will increase by which the glass-transition temperature T_{g} will increase and the polymeric constructs will further increase in strength.

Annealing also affects the crystalline domains of a polymeric material. Here, smaller crystalize segments can grow into larger segments. Hence, annealing could also increase ductility of a polymeric construct. Thereby, it will benefit to accommodate larger stretches until break than without an annealing treatment. This would impact the ability to inflate electrospun conduits when these type of structures are used for stenting applications (WO2017118755A1). In this way, the individual polymer fibers in such meshes will allow for larger plastic deformation before the polymer fibers will break when being inflated by a balloon catheter.

Furthermore, the method can comprise the following further steps:
Providing a fibrous tubular conduit made of a polymer;
providing a fibrous tubular conduit having a similar or slightly larger inner diameter compared to the crossing profile of the deflated and folded balloon catheter;
positioning the fibrous tubular conduit over that balloon catheter; and
crimping the fibrous tubular conduit onto that balloon catheter by exposing the fibrous tubular conduit to a heat treatment below its melt transition temperature.

The fibrous structure of the medical device may be received or manufacture by means of a electro-spinning process.

The fibrous structure can be a stent or a graft or the like.

The fibrous structure can be made of a bio-compatible, especially also a bio-degradable polymer.

The material for the implantable polymer stent can be biocompatible polymer. Biocompatible polymeric fiber materials can include inter alia, but not limited to it:
Bioresorbable polymers (such as poly lactic acid (PLA), including poly(L-lactide), poly(D-lactide), poly(D,L-lactide), as well as polyglycolide, polycaprolactone, polydioxanone, poly(trimethylene carbonate), poly(4-hydroxybutyrate), poly(ester amides) (PEA), polyurethanes, poly(trimethylene carbonate), poly(ethylene glycol), poly(vinyl alcohol), polyvinylpyrrolidone, and copolymers thereof such as poly(L-lactide/DL-lactide), poly(L-lactide/D-lactide), poly(L-lactide/glycolide), poly(L-lactide/caprolactone), poly(DL-lactide/glycolide),
non bioresorbable materials (such as polypropylene, polyethylene, polyethylene terephthalate, polytetrafluoroethylene, polyaryletherketone, nylon, fluorinated ethylene propylene, polybutester, and silicone, or copolymers thereof),
biological components (such as hyaluronan, collagen, gelatin, chitosan, alginate, aloe/pectin, cellulose or other biological materials originating from tissues from either autologous, allergenic or xenogenic origin),
or a combination thereof.

Advantageously, the polymeric fibers may include: poly(L-lactide), poly(D-lactide), polyglycolide or a combination thereof in the form of a co-polymer being either poly(DL-lactide), poly(lactide-co-glycolic acid) or poly(DL-lactide-co-glycolic acid).

In particular, said crystalline or semi-crystalline polymeric material may have a glass-transition temperature (T_{g}) above physiological core body temperature.

In particular, annealing allows polymer chains to relieve internal stresses, and would enable the chains to get into a more crystalline structure.

As an effect, the fibrous structure is allowed to crimp, where when applied as a tubular conduit will reduce the inner diameter. This feature can be used to crimp such tubular conduit on a balloon catheter to secure its mounting.

Further, annealing also makes these fibrous structures, e.g. tubular conduits stronger to be able to open up an artery when used as an intraluminal stent device. Hence, it also affects the amount of pressure needed to inflate the tubular conduit with the balloon catheter.

The annealing step is decisive to improve the strength and stiffness of the device.

As a tubular conduit, annealing might also have an effect on the recoil properties. Which means that after balloon inflation, the inflated conduit may to some extent return to its initial non-inflated configuration. Here, annealing of the tubular conduit might reduce the amount of recoil.

The annealing procedure can be tuned by means of but not limited to, the choice of temperature, the heating and cooling duration, the speed to heat and the speed to cool, as well as the number of cycles you want to repeat this.

To improve the mechanical properties of fibrous structures as e.g. in polymer based fibrous stents, such structures can benefit from a heat treatment, often referred to as “annealing”. Conventional crystalline polymers such as poly-lactide-acid (PLA) tend to be strong, but also brittle. Even so has PLA a relatively low glass transition temperature (T_{g}) of around 60° C.-65° C., and a melting temperature (Tₘ) around 185° C.-195° C., depending on the grade and molecular weight of the polymer. With a T_{g} above body temperature, such polymers can benefit from an annealing step by heating the polymer at or above T_{g}, but below Tₘ, which would reduce the brittle tendency of PLA and increase its ductility. Depending on the polymer composition, the glass transition temperature will deviate. As such, the T_{g} of for instance poly(DL-lactide) is around 50° C.-55° C., of polyglycolide is around 40° C.-50° C., of poly(ε-caprolactone) is around −60° C., 50/50 DL-lactide/glycolide copolymer is around 45° C.-50° C., of 85/15 L-lactide/glycolide copolymer is around 55° C.-60° C., and of 70/30 L-lactide/ε-caprolactone copolymer is around 15° C.-25° C. Despite the polymer composition, also the molecular weight of the used polymer can alter the T_{g} with a few degrees. For the avoidance of doubt, the T_{g} of a material is not a fixed single value, but instead is a range of temperatures, mostly having an optimum, in which the polymer is in a glass transition state.

During production of fibrous structures such as e.g. fibrous tubular conduits by electrospinning, polymer fibers undergo rapid solidification by solvent evaporation, in which polymer chains are locked in a conformation. This creates a buildup of stress and tension in the material and so in the polymer fiber. By heating the polymer to T_{g}, the polymer chains can move more freely, by which stresses dissipate. It is not uncommon that such an annealing treatment can increase 40% of the strength and durability and 25% of the stiffness compared to unannealed constructs. One other effect of annealing, is that due to stress relaxation in the material, the object can undergo deformation and crimp.

Furthermore, the method can comprise the following further steps:
Providing as fibrous structure a fibrous tubular conduit made of a polymer;
positioning the fibrous tubular conduit on a fixed inner carrier of similar or smaller sized outer diameter compared to inner diameter of the fibrous tubular conduit; and
constraining the fibrous tubular conduit on that fixed inner carrier by exposing the fibrous tubular conduit to a heat treatment below its melt transition temperature.

This way, a very effective crimping to inner carrier can be achieved. These steps ensure that the fibrous structure is safely and also in a balanced way mounted to the inner carrier. The inner carrier can be the balloon of the balloon catheter in case of a stent, which shall be deployed with the balloon catheter and thus must be mounted on the outside of the non-inflated balloon.

In another embodiment of the method, the method comprises the following further step that after crimping the fibrous tubular conduit to the balloon catheter annealing of the fibrous tubular conduit is continued.

By means of the continuation of the annealing the mechanical properties of the fibrous structure can be further enhanced by the mechanism of annealing as described above and at the same time the form and dimension after and due to the crimping is maintained.

Additionally, the method can comprise the following further step(s):

The fibrous structure is exposed to cycles with different temperatures, durations and repetitions of cooling and heating or any combination thereof. By this variation of thermal treatment internal (mechanical) stress in the fibrous structure can be reduced and a homogenous structure and overall internal stress level can be achieved.

Furthermore, the method can comprise the following further step(s):

The fibrous structure is exposed to heat by at least one of a convection through a gas, liquid or solid medium. This way a homogenous temperature treatment can be achieved. By means of a homogenous temperature treatment and ensuring that the temperature at the same time in all parts of the fibrous structure is more or less on the same level, homogenous mechanical properties all over the fibrous structure are received and mechanical stresses caused by inhomogenous heating or even already existing mechanical stresses are reduced or removed.

Alternatively or additionally, the method can comprise the following further step(s):

The fibrous structure is heated by infrared, which wavelength is chosen not to directly heat the material of the balloon and/or other parts of the balloon catheter. By this, only the fibrous structure is thermally treated and the properties of the fibrous structure are influenced. Further, as the balloon and also the balloon catheter is not or less thermally manipulated and heated or influenced in its temperature, it does not change or change less its dimension to due to thermal factors. This helps to achieve a high accuracy regarding the dimensions of the fibrous structure, in particular to ensure the dimensions of the stent in terms of its diameter and length.

Furthermore, the present invention relates to a medical device. Accordingly, the medical device comprises a fibrous structure, wherein the fibrous structure is annealed.

The medical device can received by means of the method or a method according to one of the described possible embodiments, especially as described above. Further details will now be explained in the drawings. It is shown in

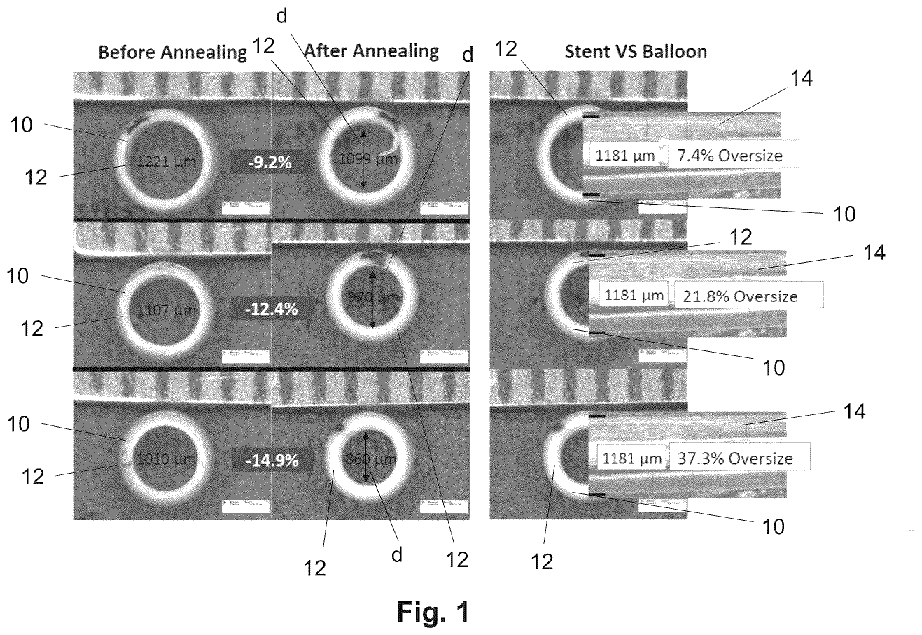

FIG. 1 the effect of the annealing according to an embodiment of the present invention;

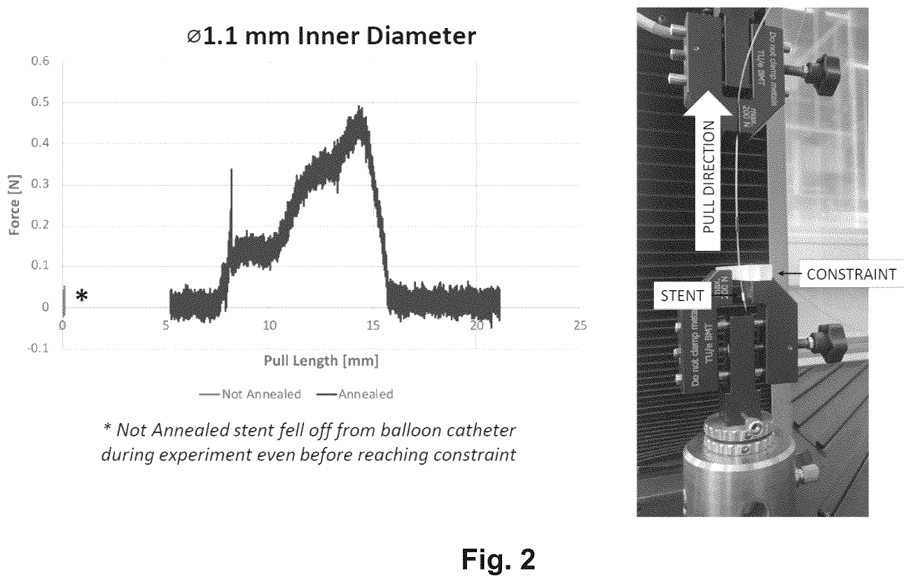

FIG. 2 the effect of the dislodgement force in connection with the annealing as shown in FIG. 1.

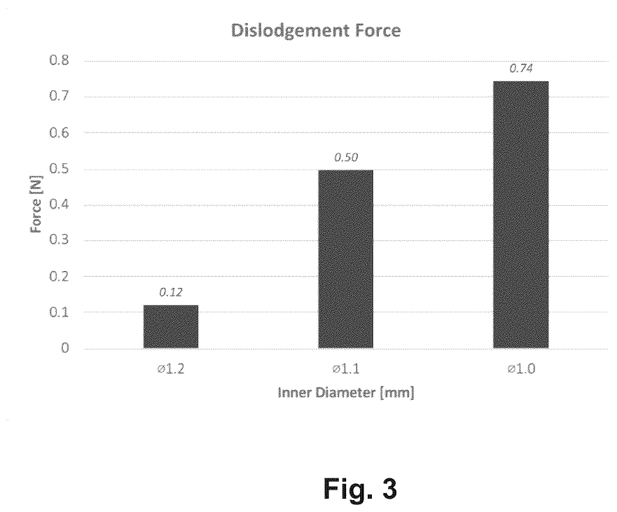

FIG. 3 the dislodgement force of stents with an initial inner diameter closer to the outer diameter of the deflated balloon in connection with the annealing from the stent with the initial inner diameter of 1.1 mm (or 1107 μm) as shown in FIG. 1;

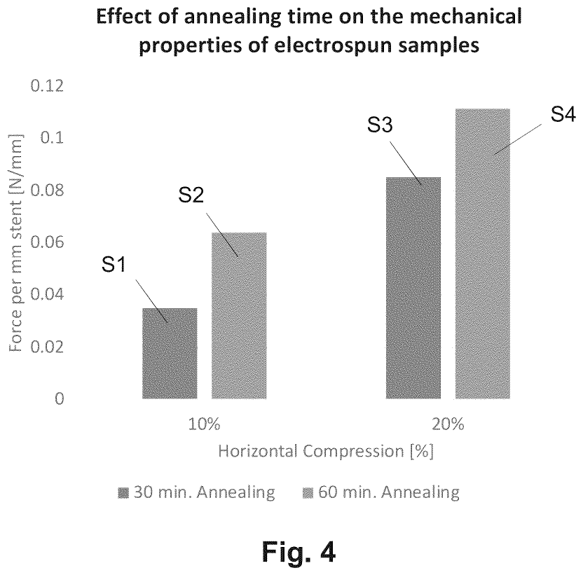

FIG. 4 a diagram related to the effect of annealing time on the mechanical properties of electropunk samples;

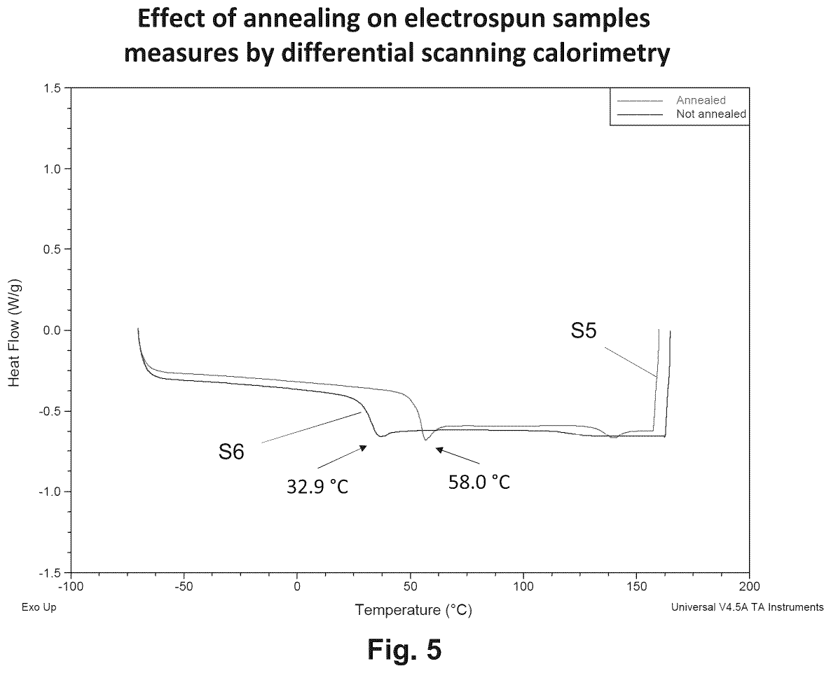

FIG. 5 a diagram related to the effect of annealing on electrospun samples measures by differential scanning calorimetry; and

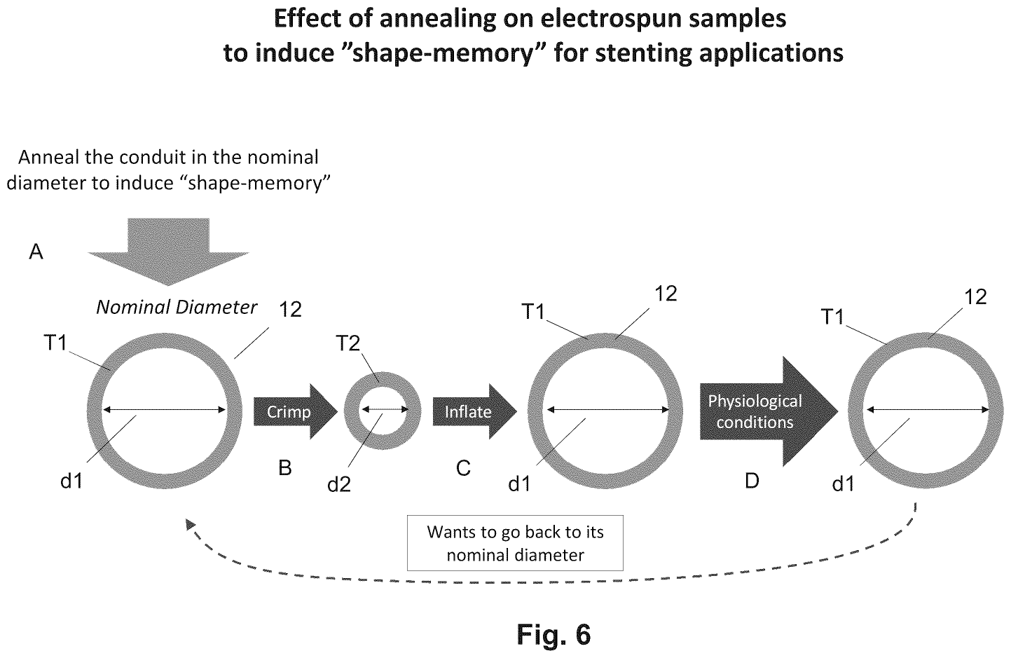

FIG. 6 a schematic drawing of the effect of annealing on electrospun samples to induce “shape-memory for stenting applications.

FIG. 1 shows the effect of the annealing according to an embodiment of the present invention.

Three examples of an electrospun fibrous structure are shown to illustrate the effect of annealing.

The term “stent” refers to a structure that provides support to the vascular wall.

The term “annealing” refers to a heat treatment in which the implant may improve in strength, toughness, hardness and ductility.

The term “bioabsorbable”, “biodegradable”, “bioresorbable” refers to the ability of a material to be decomposed and eliminated by the body.

The shown embodiment of the invention is here a fibrous structure 10, e.g. a tubular conduit forming a medical device, i.e. an implantable stent 12.

This stent is intended for use and deployment in e.g. a cardiovascular vessel or any other other vessel of a patient.

The tubular conduit, i.e. the stent 12 is composed of fibers, preferably in the micro and/or nanometric range.

These fibers may be composed of a bioabsorbable material.

In particular, the fibrous structure can be made of a bio-compatible, especially also a bio-degradable polymer.

The material for the fibrous structure, for example for an implantable polymer stent, can be biocompatible polymer. Biocompatible polymeric fiber materials can include inter alia, but not limited to it:
Bioresorbable polymers (such as poly lactic acid (PLA), including poly(L-lactide), poly(D-lactide), poly(D,L-lactide), as well as polyglycolide, polycaprolactone, polydioxanone, poly(trimethylene carbonate), poly(4-hydroxybutyrate), poly(ester amides) (PEA), polyurethanes, poly(trimethylene carbonate), poly(ethylene glycol), poly(vinyl alcohol), polyvinylpyrrolidone, and copolymers thereof such as poly(L-lactide/DL-lactide), poly(L-lactide/D-lactide), poly(L-lactide/glycolide), poly(L-lactide/caprolactone), poly(DL-lactide/glycolide),
non bioresorbable materials (such as polypropylene, polyethylene, polyethylene terephthalate, polytetrafluoroethylene, polyaryletherketone, nylon, fluorinated ethylene propylene, polybutester, and silicone, or copolymers thereof),
biological components (such as hyaluronan, collagen, gelatin, chitosan, alginate, aloe/pectin, cellulose or other biological materials originating from tissues from either autologous, allergenic or xenogenic origin),
or a combination thereof.

Advantageously, the polymeric fibers may include: poly(L-lactide), poly(D-lactide), polyglycolide or a combination thereof in the form of a co-polymer being either poly(DL-lactide), poly(lactide-co-glycolic acid) or poly(DL-lactide-co-glycolic acid).

In particular, said crystalline or semi-crystalline polymeric material may have a glass-transition temperature (T_{g}) above physiological core body temperature.

The fibers form a network of layers of stacked fibers.

The fibers can form a network which is either randomly organized or composed of aligned fibers, or a combination thereof.

The fibrous conduit can be made by a fiber forming production method such as electrospinning, in which fibers can be collected onto a rod, resulting in a tubular conduit composed by a fibrous network wall. Possible ways of achieving such a fibrous conduit are e.g. disclosed in U.S. Pat. No. 10,813,777 B2. The fibrous structure is preferably made of a network of polymeric fibers in the micrometric or nanometric range.

The outer diameter of this rod may be chosen to be just slightly larger than the crossing profile of the folded delivery balloon.

After production of the fibrous tubular conduit, the tube needs to be cut at a specific length fitting the balloon.

Hereafter, the fibrous tube, i.e. the stent 12, was positioned onto the delivery balloon 14.

The stent 12 will be exposed to an elevated temperature in range or above the glass transition temperature of the polymer of which the fibrous tube is made of, but below the melt transition temperature of that polymer.

In the shown embodiment, the following process parameters were applied:

The heat was applied using a conventional pre-heated hot air circulation oven at atmospheric room pressure

The set temperature was 65° C.

The duration was 1 hour.

Samples where instantly exposed to the heat in the over, and when taken out after 1 hour, and allowed to cool down on a lab bench at room temperature.

No repeated annealing cycles where applied.

This way, a medical device, here an implantable stent 12 was provided with a method comprising the steps of:
Providing a fibrous structure; and
annealing said fibrous structure.

Usual temperature ranges to anneal the material of interest for stent applications are above body temperature and below the melting temperature, and ideally around the glass transition temperature of that material. Usually the annealing takes up a few minutes to hours to reach a steady state. Long exposure to elevated heat might result in polymer decomposition and should be avoided. Several few repeating heating and cooling cycles might be applicable.

This elevated temperature can be maintained for a certain period of time until polymer reorganization in response to the elevated temperature has been completed, or may be interrupted at any preferred point in time.

The elevated temperature might be applied in cycles composed of cooling and heating steps in which the time duration of each step may be altered.

While heating the polymer to the glass transition temperature, the tubular conduit will crimp for which the inner diameter of the tube will become smaller.

As a consequence, the fibrous tube will fixate itself onto the balloon catheter when choosing a starting inner diameter only slightly larger than the deflated balloon of choice.

So, the steps as follows were also conducted:
providing a fibrous tubular conduit made of a polymer and;
providing a fibrous tubular conduit having a similar or slightly larger inner diameter compared to the crossing profile of the deflated and folded balloon catheter;
positioning the fibrous tubular conduit over that balloon catheter;
crimping the fibrous tubular conduit onto that balloon catheter by exposing the fibrous tubular conduit to a heat treatment below its melt transition temperature.

It is possible that the annealing process is continued after the crimping.

FIG. 1 shows the structure before annealing, after annealing and the structure, i.e. the stent 12, with respect to the balloon 14.

The balloon 14 is the balloon of the balloon catheter, with which the stent 12 will be deployed.

By doing so, the dislodgement force which will be needed to slide the stent from the balloon after being exposed to such an annealing step will increase for which the stent 12 with its inner diameter d will be more firmly attached to the balloon catheter when coming closer in range to the outer diameter of the deflated balloon of choice.

FIG. 2 shows the effect of the dislodgement force in connection with the annealing. Using a uniaxial tensile tester, the stent mounted on the balloon is being pulled through a constraint. The amount of force needed to pull the stent from the balloon is being measured in this way. Here, it was seen that the unannealed stent with an initial 1.1 mm inner diameter simply fell off from the balloon even before reaching the constraint, as it was too loose on the balloon catheter.

The annealed stent with an initial 1.1 mm inner diameter instead was mounted firmly and required 0.5 N to be pulled from the balloon catheter. This confirmed that annealing of the stent on the balloon catheter improved the dislodgement force.

FIG. 3 shows that the dislodgement force can even be further improved by producing stents with an initial inner diameter closer to the outer diameter of the balloon after annealing.

In one embodiment heat may be applied to the fibrous tube by heat convection by air in for instance a heating oven, or by liquid.

In another embodiment the part of the fibrous tube mounted on the balloon, excluding the catheter wire, might be placed on a conductive mold to locally heat the polymer.

In yet another embodiment the polymer fibrous tube might be exposed to infrared light, in which the wavelength in optimized to only heat the polymer of the fibrous tube, but not the material of the balloon catheter.

The heat treatment of the fibrous tube does not have to be uniform over the fibrous tube.

In one embodiment the extremities of the fibrous tube might undergo a different heat treatment than the center of the tube. In yet another embodiment selective heat patterns may be applied to the fibrous implant.

In addition to making use of the ability to make the fibrous tube crimp in diameter when applying a heating step, a consequential effect is that the fibrous tube will increase in stiffness.

Thereby the fibrous tubes can withhold larger externally applied mechanical loads after the annealing step than before the annealing step. In this way the strength of the fibrous tube can be tuned. In one embodiment the fibrous tube is used as a stent in a clinical setting in which the intended use is to open a stenotic lesion.

Depending on the severity of the stenosis, the externally applied mechanical load by this lesion should be below the maximum externally applied load the stent can withstand, but can be adjusted by tuning the annealing procedure of this fibrous tube during manufacturing.

FIG. 4 shows a diagram related to the effect of annealing time on the mechanical properties of electropunk samples.

Here the increase of mechanical crush properties by prolonging the heating period of the annealing procedure from 30 minutes to 60 minutes. Prolonging the heating period resulted in elevated crush force when exposing the electrospun structures to horizontal crush compression of either 10% and 20%.

The left side shows the effect of 30 minutes (sample 1=S1) compared with 60 minutes (sample 2=S2) of annealing with a horizontal compression of 10%. The crush is elevated from a level of approx. 0.035 N/mm (30 min. annealing and 10% horizontal compression) to more than 0.06 N/mm (60 min. annealing and 10% horizontal compression).

The right side shows the effect of 30 minutes compared with 60 minutes of annealing with a horizontal compression of 20%.

The right side shows the effect of 30 minutes (sample 3=S3) compared with 60 minutes (sample 4=S4) of annealing with a horizontal compression of 20%. The crush is elevated from a level of approx. 0.085 N/mm (30 min. annealing and 20% horizontal compression) to more than 0.11 N/mm (60 min. annealing and 20% horizontal compression).

One can see that the effect of annealing can be further enhanced by compression of the electrospun structure, see also FIG. 6.

FIG. 5 shows a diagram related to the effect of annealing on electrospun samples measures by differential scanning calorimetry.

Here, an increase in the T_{g} of electrospun structures (here electrospun conduits) after being exposed to an annealing step reaching 58.0° C. can be seen in sample S5, when compared to the non-annealed sample (sample S6) having a T_{g} at 32.9° C. as was measured by differential scanning calorimetry.

FIG. 6 shows a schematic drawing of the effect of annealing on electrospun samples to induce “shape-memory for stenting applications.

In FIG. 6 a method is disclosed how annealing may be used to induce shape-memory. This will be beneficial for stent application, where in case of crimping of compressing the conduit for implantation purposes, it wants to go back to its shape memory configuration upon positioning of the implant to limit recoil of the stent.

The electrospun sample is here a stent 12, which has after electrospinning in Step A the nominal diameter. It is in this state annealed to induce a “shape-memory”.

Then, after annealing the stent 12 (basically being a tubular conduit with a diameter d1 and a wall thickness T1) is reduced to a smaller diameter by means of crimping in Step B, resulting in a stent structure which is ready for deployment with a reduced diameter d2, but an increased wall thickness T2. The wall thickness T2 is larger than the wall thickness T1. Usually, this step of crimping is performed such that the stent 12 placed on the non-inflated balloon of the balloon catheter, with which the stent 12 shall be deployed.

In Step C the stent 12 is deployed by inflating, e.g. implanted into a blood vessel. There it is subject to a temperature of approx. 36-37° C. and fully wetted.

In such conditions the material tends to go back to its nominal diameter, which will result in the situation that the stent 12 will extend itself against the inner vessel wall and so a self-fixating effect is gained as shown in Step D.

### REFERENCES

10 fibrous structure
12 stent
14 balloon (of balloon catheter)
d inner diameter of fibrous structure
S1 sample 1
S2 sample 2
S3 sample 3
S4 sample 4
S5 sample 5
S6 sample 6
d1 diameter
d2 diameter
T1 wall thickness
T2 wall thickness
A step A
B step B
C step C
D step D

1. A method of making a medical device comprising:
providing a fibrous structure; and
annealing said fibrous structure.

2. The method of claim 1,
wherein
the method comprises the following further steps:
providing a fibrous tubular conduit made of a polymer and;
providing a fibrous tubular conduit having a similar or slightly larger inner diameter compared to the crossing profile of the deflated and folded balloon catheter;
positioning the fibrous tubular conduit over that balloon catheter; and
crimping the fibrous tubular conduit onto that balloon catheter by exposing the fibrous tubular conduit to a heat treatment below its melt transition temperature.

3. The method of claim 1,
wherein
the method comprises the following further steps:
providing as fibrous structure a fibrous tubular conduit made of a polymer; and
positioning the fibrous tubular conduit on a fixed inner carrier of similar or smaller sized outer diameter compared to inner diameter of the fibrous tubular conduit; and
constraining the fibrous tubular conduit on that fixed inner carrier by exposing the fibrous tubular conduit to a heat treatment below its melt transition temperature.

4. The method according to claim 2,
wherein
the method comprises the following further step:
after crimping the fibrous tubular conduit to the balloon catheter annealing of the fibrous tubular conduit is continued.

5. The method according to claim 1,
wherein
the method comprises the following further step(s):
the fibrous structure is exposed to cycles with different temperatures, durations and repetitions of cooling and heating or any combination thereof.

6. The method according to claim 1,
wherein
the method comprises the following further step(s):
the fibrous structure is exposed to heat by at least one of a convection through a gas, liquid or solid medium.

7. The method according to claim 1,
wherein
the method comprises the following further step(s):
the fibrous structure is heated by infrared, which wavelength is chosen not to directly heat the material of the balloon and/or other parts of the balloon catheter.

8. A medical device comprising a fibrous structure, wherein the fibrous structure is annealed.

9. (canceled)
